(19) United States
(12) Patent Application Publication
(10) Pub. No.: US 2013/0253312 A1
(43) Pub. Date: Sep. 26, 2013

(54) MEDICAL TOOL THAT EMITS NEAR INFRARED FLUORESCENCE AND MEDICAL TOOL POSITION-CONFIRMING SYSTEM

(71) Applicants: Takayuki Sato, Kochi (JP); Katsumi Noguchi, Tokyo (JP)

(72) Inventors: Takayuki Sato, Kochi (JP); Katsumi Noguchi, Tokyo (JP)

(73) Assignee: National University Corporation Kochi University, Kochi (JP)

(21) Appl. No.: 13/990,468

(22) PCT Filed: Nov. 24, 2011
(86) PCT No.: PCT/JP2011/076995

§ 371 Date: May 30, 2013

(30) Foreign Application Priority Data
Dec. 2, 2010 (JP) 2010-268934

(51) Int. Cl. A61B 5/00; A61M 27/00; A61B 1/06; A61B 1/00; A61B 5/06; A61B 1/04

(52) U.S. Cl. CPC A61B 5/0086; A61B 5/064; A61B 1/043; A61B 1/06; A61B 1/00045; A61M 27/00; A61B 5/0071
USPC 600/424

(57) ABSTRACT

A medical tool position-confirming system is provided with a medical tool 1 capable of emitting light, the medical tool 1 including a luminescent agent emitting near infrared fluorescence when irradiated with near infrared light having a wavelength within the range of 600 to 1400 nm, the luminescent agent being applied on a surface of the medical tool or kneaded into the medical tool, a light source 3 for directing the near infrared light 2 toward the medical tool 1, a camera 4 for receiving the near infrared fluorescence emitted from the luminescent agent of the medical tool 1, and a monitor 6 for displaying an image 5 taken by the camera 4. The position of the medical tool such as a shunt tube can be confirmed without using an X-ray.

### TECHNICAL FIELD

The present invention relates to a medical tool that emits near infrared fluorescence when irradiated with near infrared light, and a medical tool position-confirming system that is capable of confirming the position of the medical tool by receiving near infrared fluorescence emitted from the medical tool.

### BACKGROUND ART

Health care professionals have a desire to confirm the position, inside the body, of a medical tool (a stent, an embolization coil, a catheter tube, an injection needle, a shunt tube, a drain tube, an implant and the like) to be inserted into or placed in the body of a patient. For example, when inserting an injection needle into a specific blood vessel for dialysis or the like, there is a desire to confirm, during the operation, as to whether or not the injection needle is certainly inserted into the target blood vessel.

Conventionally, the confirmation of the position of a medical tool as described above has been generally performed by applying an X-ray to a patient and observing a perspective image thereof.

Further, there has been proposed a method, as means for confirming the traveling direction and the position of a catheter which has been inserted into a patient, in which a catheter is configured into an optical waveguide catheter by attaching an optical fiber to the catheter to thereby cause the distal end of the catheter to emit light. More specifically, near infrared light is caused to be incident on the catheter, and the near infrared light emitted from the distal end thereof is detected by a photodetector from the outside of the body of a patient, thereby detecting the position of the distal end of the catheter (see Patent Literature 1, for example).

### CITATION LIST

### Patent Literature

Patent Literature 1: JP 2010-528818 W

### SUMMARY OF INVENTION

### Technical Problem

In a method using an X-ray to confirm the position of a medical tool, there is a problem in that an operator and a patient are continuously exposed to X-ray radiation during the operation. In addition, there is a problem in that the method is difficult to be utilized in a medical tool that is made of an X-ray transmissive resin.

Further, in a method using an optical waveguide catheter, there is a problem in that only a point of the catheter such as the distal end thereof can be detected, and it is therefore difficult to detect the position, the direction, the posture and the like of the entire medical tool.

It is therefore an object of the present invention to provide a medical tool that is capable of solving the above problems and a position detection system for the medical tool.

### Solution to Problem

In order to solve the above problems, the present invention employs the following configurations.

Although, the reference numerals in the drawings are provided in parentheses for facilitating the understanding of the present invention, the present invention is not limited thereto.

Specifically, a first aspect of the present invention employs a medical tool (1) that emits near infrared fluorescence, the medical tool including a luminescent agent emitting near infrared fluorescence when irradiated with near infrared light (2) having a wavelength within the range of 600 to 1400 nm, the luminescent agent being applied on a surface of the medical tool (1) or kneaded into the medical tool (1).

In a second aspect of the present invention, a main body of the medical tool (1) emitting near infrared fluorescence may be a shunt tube, and the luminescent agent may be applied on an entire surface of the main body or kneaded into the entire main body in the medical tool (1) of the first aspect of the present invention.

Further, a third aspect of the present invention employs a medical tool position-confirming system including a medical tool (1) capable of emitting light, the medical tool (1) including a luminescent agent emitting near infrared fluorescence when irradiated with near infrared light (2) having a wavelength within the range of 600 to 1400 nm, the luminescent agent being applied on a surface of the medical tool (1) or kneaded into the medical tool (1), a light source (3) for directing the near infrared light (2) toward the medical tool (1), a camera (4) for receiving the near infrared fluorescence emitted from the luminescent agent of the medical tool (1), and a monitor (6) for displaying an image (5) taken by the camera (4).

In a fourth aspect of the present invention, a main body of the medical tool (1) maybe a shunt tube, and the luminescent agent may be applied on an entire surface of the main body or kneaded into the entire main body in the medical tool position-confirming system of the third aspect of the present invention.

### Advantageous Effects of Invention

The present invention makes it possible to easily and safely detect the position of the medical tool (1) without using an X-ray, and therefore possible to prevent radiation exposure to a patient and a health care professional. In addition, it is possible to easily and accurately confirm the position, the direction, the posture and the like of the entire medical tool (1).

### BRIEF DESCRIPTION OF DRAWINGS

FIG. 1 is an explanatory drawing of a medical tool position-confirming system according to the present invention.

FIG. 2 is an explanatory drawing illustrating a state in which the position of a shunt tube which has been inserted into a body of a patient is being confirmed.

FIG. 3 is an explanatory drawing illustrating a state in which the position of a stent which has been inserted into a blood vessel of a patient is being confirmed.

FIG. 4 is an explanatory drawing illustrating a state in which the position of an injection needle which has been inserted into a blood vessel of a patient is being confirmed.

FIG. 5 is an explanatory drawing illustrating a state in which the position of a catheter which has been inserted into a blood vessel of a patient is being confirmed.

### DESCRIPTION OF EMBODIMENTS

Hereinafter, embodiments of the present invention will be described with reference to the drawings.

### Embodiment 1

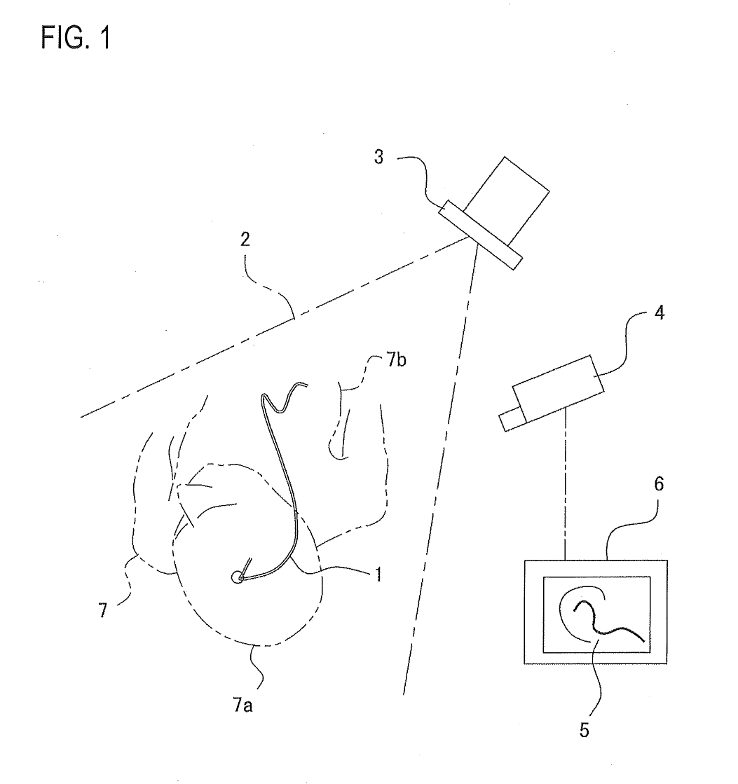

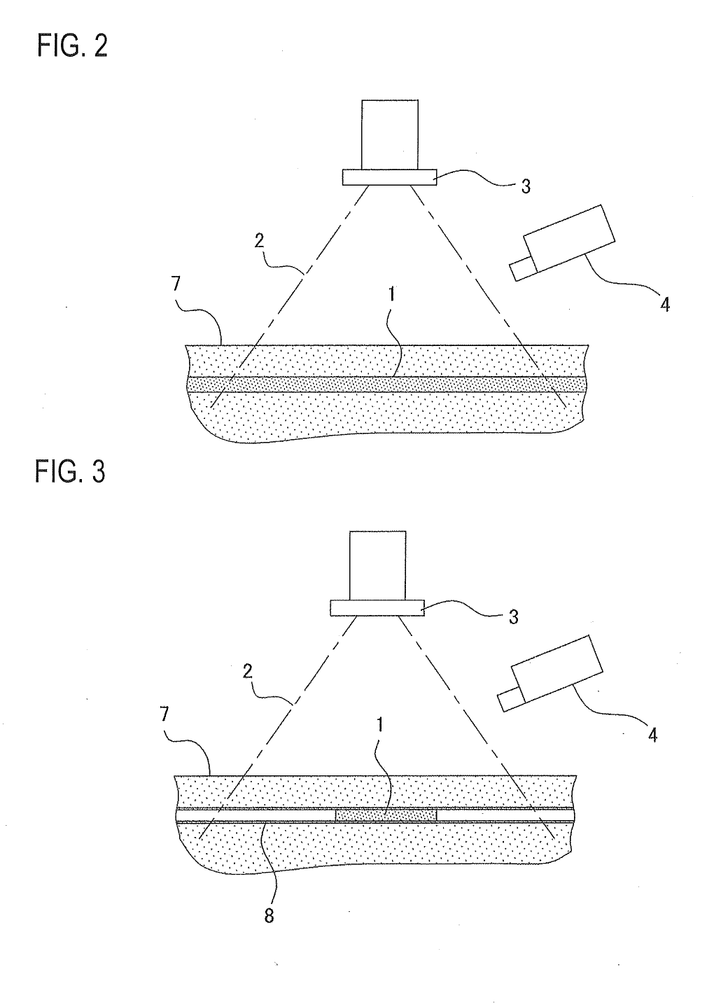

As shown in FIGS. 1 and 2, in a medical tool position-confirming system, there are used a medical tool 1 which emits near infrared fluorescence, a light source 3 which directs near infrared light 2 toward the medical tool 1, a camera 4 which receives fluorescence emitted from a luminescent agent of the medical tool 1, and a monitor 6 which displays an image 5 of the medical tool 1, the image 5 being taken by the camera 4.

In particular, the medical tool 1 is a shunt tube. The luminescent agent which emits near infrared fluorescence when irradiated with the near infrared light 2 having a wavelength within the range of 600 to 1400 nm is applied on a surface of the shunt tube. Alternatively, the luminescent agent is previously kneaded into a resin that is a constituent material of the shunt tube which is the medical tool 1.

For example, indocyanine green which emits light when irradiated with near infrared light can be used as the luminescent agent. It is needless to say that the luminescent agent is an agent that is usable in a human body or an animal.

A LED which emits the near infrared light 2 is used as the light source 3. The near infrared light 2 relatively easily penetrates a human body 7, and can penetrate the human body 7 up to approximately 5 to 20 mm deep from a tissue surface thereof. Further, the near infrared light 2 preferably has a wavelength in a range that is suitable for causing the luminescent agent to emit light. Therefore, near infrared light having a wavelength within the range of 600 to 1400 nm can be used as the near infrared light 2. When the luminescent agent is indocyanine green, the wavelength range is preferably 700 to 1100 nm.

When the near infrared light 2 is directed from the light source 3 toward a region of the human body 7 into which the medical tool 1 is inserted to 5 to 20 mm deep from a tissue surface thereof, the medical tool 1 emits near infrared. fluorescence.

The camera 4 can take an image of the entire medical tool 1 by receiving the near infrared fluorescence emitted from the luminescent agent of the medical tool 1 by a light receiving element. More specifically, the near infrared fluorescence emitted from the luminescent agent of the medical tool 1 penetrates tissue of the human body 7, and the camera 4 receives the penetrating near infrared fluorescence to thereby take an image of the medical tool 1. At the same time, an image of an outline or the like of the human body 7 is also taken by the camera 4.

The image taken by the camera 4 may be a monochromatic image, or may also be a colored image. Further, the light source 3 may be light sources annularly arranged around a lens of the camera 4. This makes it possible to more properly take an image of the medical tool 1.

The image 5 of the medical tool 1 taken by the camera 4 is displayed on a projection surface of the monitor 6 as a monochromatic or colored image. A health care professional such as an operator can confirm the position, the posture, the direction and the like of the medical tool 1 inside the human body 7 by viewing the image 5 of the medical tool 1 displayed on the monitor 6.

Next, the operation of the medical tool position-confirming system having the above configuration will be described.

(1) As shown in FIGS. 1 and 2, a shunt tube as the medical tool 1 which emits near infrared fluorescence is inserted into the body of a patient by an operator.

The shunt tube is passed under the skin of a patient and used to drain cerebrospinal fluid therethrough into the abdominal cavity in treatment for hydrocephalus. The shunt tube is passed from a head 7a to an abdomen 7b of the patient at a depth of 5 to 20 mm from the epidermis.

(2) While the shunt tube as the medical tool 1 is being inserted into the body of the patient, the near infrared light 2 having a wavelength in the above range is directed to the patient from the light source 3.

When the near infrared light 2 from the light source 3 falls on the medical tool 1 which is located under the epidermis of the patient, the entire medical tool 1 emits near infrared fluorescence. The emitted near infrared fluorescence is received by the camera 4, and a whole image of the medical tool 1 is displayed on the monitor 6. Further, light reflected by the surface of the human body 7 is also received by the camera 4, and an image of the outline of the human body 7 is thereby also displayed on the monitor 6 together with the image of the medical tool 1.

(3) A health care professional such as an operator performs an operation or treatment while confirming the travelling direction of the tip of the medical tool 1 and the position, the posture, the direction and the like of the entire medical tool 1 by viewing the images of the medical tool 1 and the human body 7 displayed on the monitor 6.

Accordingly, during the operation and subsequent medical procedure for the patient with hydrocephalus, it is possible to confirm a route under the skin to the inside of the abdominal cavity along which the shunt tube is passed through and placed without using an X-ray.

### Embodiment 2

As shown in FIG. 3, a medical tool 1 used in an embodiment 2 is a stent to be inserted into a blood vessel 8.

In the embodiment 2, during craniotomy procedure for cerebral aneurysm, a stent having a luminescent agent which is applied thereon or kneaded thereinto is used as an endovascular approach in combination with a surgical approach. Alternatively, during thoracotomy procedure, a stent having a luminescent agent which is applied thereon or kneaded thereinto is used as an endovascular approach in combination with a surgical approach.

During the surgical procedure, near infrared light 2 is directed toward an affected part from a light source 3 from the outside of a body, the medical tool 1 located inside the affected part thereby emits fluorescence, and the emitted fluorescence is received by a camera 4. As a result, a whole image of the stent is displayed on a monitor 6 in real time.

In this manner, during the surgical procedure, an operator or the like can perform the surgical procedure while easily confirming the position, the posture and the like of the stent as the medical tool 1 from the outside of the blood vessel 8 or the surface of an organ without using an X-ray.

### Embodiment 3

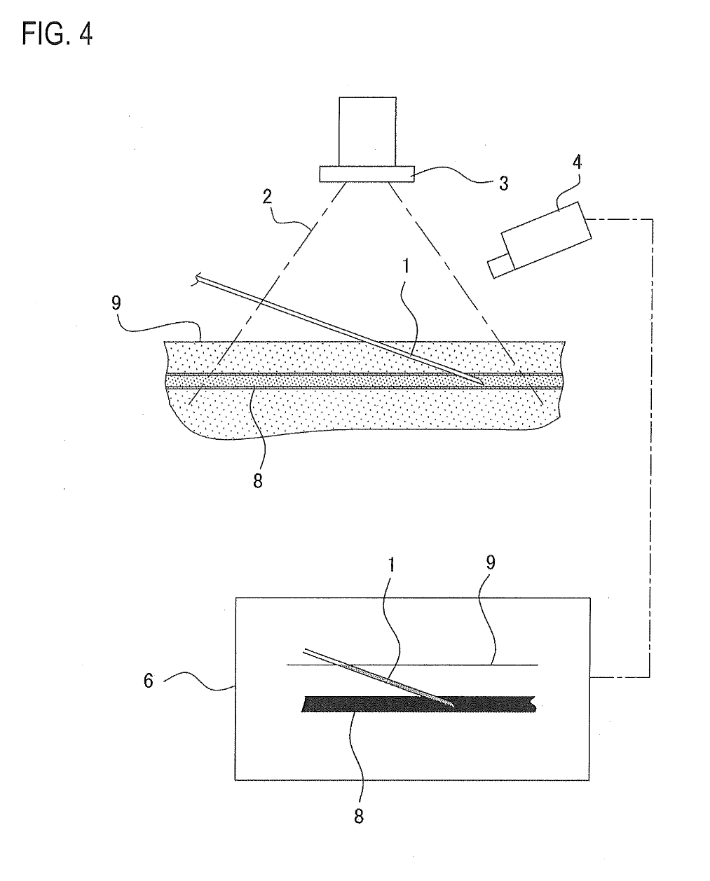

As shown in FIG. 4, a medical tool 1 emitting near infrared fluorescence which is used in an embodiment 3 is an injection needle to be inserted into a blood vessel 8.

In the embodiment 3, the injection needle as the medical tool 1 is stuck into a vein of a patient from a skin 9. The luminescent agent is applied onto a surface of the injection needle.

During the medical procedure with the injection needle, near infrared light 2 is directed toward an affected part from a light source 3 through the skin 9, the injection needle located under the skin of the patient thereby emits fluorescence, and the emitted fluorescence is received by a camera 4. As a result, a whole image including the injection needle and the blood vessel 8 which is a vein is displayed on a monitor 6 in real time. In this case, since reduced hemoglobin in venous blood absorbs a near infrared light component having a wavelength within the range of 600 to 800 nm, the vein is displayed in black on the monitor 6. Since near infrared fluorescent color emitted from the surface of the injection needle as the medical tool 1 penetrates the skin to the outside thereof, the injection needle is displayed in near infrared fluorescent color on the monitor 6.

In this manner, an operator or the like can easily and accurately insert the injection needle into the vein while confirming the positional relationship between the blood vessel 8 and the injection needle from the outside of the skin 9 of the patient without using an X-ray.

According to experiments by the present inventors, it is possible to visually recognize an injection needle and a blood vessel even at a deep part located approximately 2 cm from the skin.

### Embodiment 4

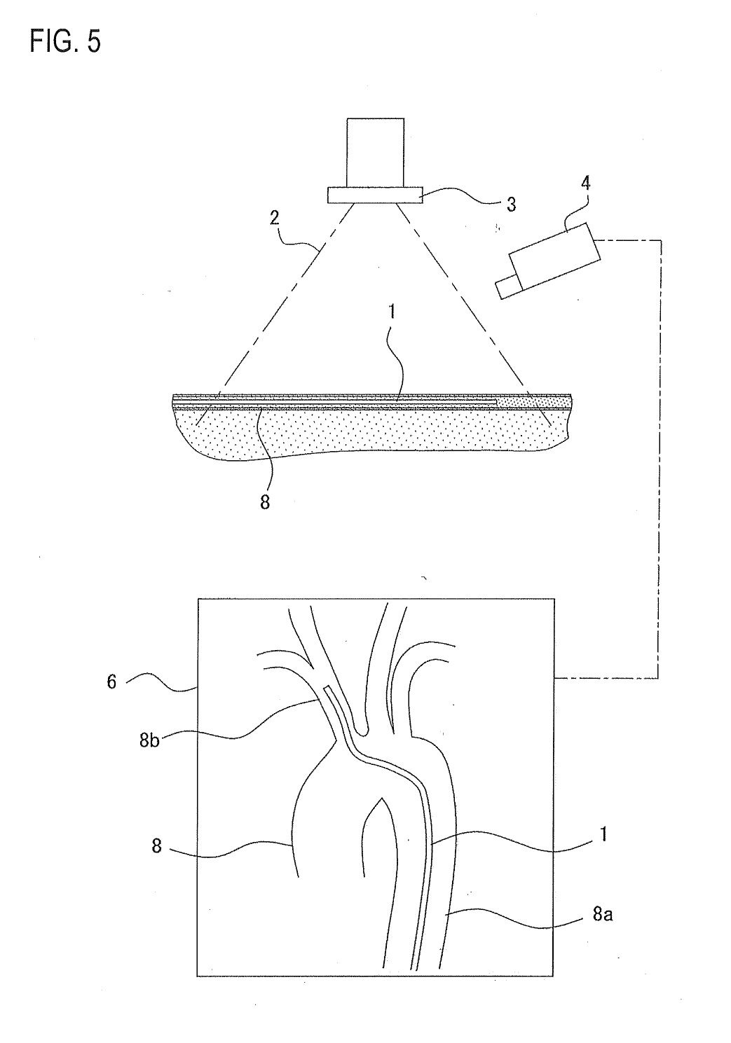

As shown in FIG. 5, a medical tool 1 emitting near infrared fluorescence which is used in an embodiment 4 is a catheter to be inserted into a blood vessel 8.

The luminescent agent is applied onto a surface of the catheter, or kneaded into a material of the catheter.

During thoracotomy procedure, when the catheter is inserted into the blood vessel 8 which has been exposed, near infrared light 2 is directed toward the exposed blood vessel 8 from a light source 3, the catheter located inside the blood vessel 8 of a patient thereby emits fluorescence, and the emitted fluorescence is received by a camera 4. As a result, a whole image including the catheter and the blood vessel 8 is displayed on a monitor 6 in real time. In this case, since near infrared fluorescent color emitted from the surface of the catheter as the medical tool 1 penetrates the blood vessel 8 to the outside thereof, the catheter is displayed in near infrared fluorescent color on the monitor 6.

In the embodiment 4, the blood vessel 8 is an artery, and the catheter is being inserted into a carotid artery from a main artery. In these arteries, reference numeral 8a designates a descending aorta and reference numeral 8b designates a brachiocephalic artery.

In this manner, an operator or the like can easily and accurately insert the catheter into the artery while confirming the positions of the tip and the entire body of the catheter, the travelling direction of the tip and the like inside the blood vessel 8 from the outside of the blood vessel 8 of the patient to without using an X-ray.

Further, the present invention is not limited to the above embodiments, and can be applied to the following embodiments.

(1) For example, although the operations for hydrocephalus and a blood vessel, and the use of a shunt tube and a stent have been described in the above embodiments, the present invention can also be applied to other operations and medical tools. For example, the present invention can also be applied, as a medical tool, to an embolization coil, a catheter tube, a drain tube, and an implant.

(2) Further, when a stent is placed in a bile duct or a pancreatic duct during laparoscopic surgery, the position, the posture, the direction and the like of the stent can be confirmed from the outside of a blood vessel or the surface of an organ without using an X-ray. Further, when a catheter is inserted into a vein, infrared light having a wavelength that is suitable for a luminescent agent is applied thereto together with infrared light having a wavelength that is easy to be absorbed by the vein, and images are taken by a camera, thereby confirming the position of the vein and the position of an injection needle or the catheter as a medical tool at the same time. As a result, it is possible to confirm as to whether or not the injection needle or the catheter is accurately inserted into the vein.

(3) Further, when a cannula of a dialysis needle is removed from a blood vessel, the tip of the cannula may be detached and left inside the blood vessel. However, even in such a case, when the cannula has a luminescent agent which is applied thereon or the like, the position in which the detached tip of the cannula is left under the skin can be easily and accurately identified, thereby making it possible to perform non-invasive treatment.

(4) The present invention can be applied not only to a medical practice for humans, but also to a medical care for animals.

(5) By placing a tube or the like inside the body of a patient, and then injecting and administering a fluorescent reagent as a luminescent agent into the tube or the like, the tube or the like can be identified without using an X-ray, an electron beam or the like.

### EXPLANATION OF REFERENCE NUMERALS

1 medical tool

2 near infrared light

3 light source

4 camera

5 image

6 monitor

1. A medical tool that emits near infrared fluorescence, the medical tool comprising a luminescent agent emitting near infrared fluorescence when irradiated with near infrared light having a wavelength within the range of 600 to 1400 nm, the luminescent agent being applied on a surface of the medical tool or kneaded into the medical tool.

2. The medical tool that emits near infrared fluorescence according to claim 1, wherein a main body of the medical tool is a shunt tube, and the luminescent agent is applied on an entire surface of the main body or kneaded into the entire main body.

3. A medical tool position-confirming system comprising:
a medical tool capable of emitting light, the medical tool including a luminescent agent emitting near infrared fluorescence when irradiated with near infrared light having a wavelength within the range of 600 to 1400 nm, the luminescent agent being applied on a surface of the medical tool or kneaded into the medical tool;
a light source for directing the near infrared light toward the medical tool;
a camera for receiving the near infrared fluorescence emitted from the luminescent agent of the medical tool; and
a monitor for displaying an image taken by the camera.

4. The medical tool position-confirming system according to claim 3, wherein a main body of the medical tool is a shunt tube, and the luminescent agent is applied on an entire surface of the main body or kneaded into the entire main body.
